(19) United States
(12) Patent Application Publication
(10) Pub. No.: US 2012/0238830 A1
(43) Pub. Date: Sep. 20, 2012

(54) Hybrid Fiber-Optic Cannula

(75) Inventors: Mirko Vukeljic, Qubec (CA); Jean-Luc Neron, L'Ancienne-Lorette (CA); Sead Doric, L'Ancienne-Lorette (CA)

(73) Assignee: DORIC LENSES INC., L'Ancienne-Lorette (CA)

(21) Appl. No.: 13/303,814

(22) Filed: Nov. 23, 2011

Related U.S. Application Data
(63) Continuation-in-part of application No. 13/046,904, filed on Mar. 14, 2011.

(51) Int. Cl. A61B 1/06; B32B 37/12; B32B 37/14; B23P 17/04

(52) U.S. Cl. 600/249; 29/428; 156/60

(57) ABSTRACT

The hybrid fiber-optic cannula can have a body having an implant end, a light passage extending through the body, and a light inlet end coinciding with the light passage, an optical fiber held in the body in coincidence with the light passage and oriented out the implant end, the light inlet end being opposite the implant end relative to the light passage, and a conduit extending through the body between a conduit outlet located at the implant end and a conduit inlet. A fluid and/or electrical wires, can be conveyed by the conduit, for instance.

### CROSS-REFERENCE TO RELATED APPLICATIONS/PRIORITY CLAIM

This application is a Continuation-in-Part of U.S. application Ser. No. 13/046,904, entitled “Optogenetic Fiber Optic Cannula and Adapted Fiber Optic Connector”, filed Mar. 14, 2011, claiming priority of U.S. provisional application 61/313,258, filed Mar. 12, 2010, the contents of both of which are hereby incorporated by reference.

### FIELD

The improvements generally relate to the communication of a light signal between a body tissue and a location external to the body. More particularly, this specification describes a unitary cannula combining a conduit for a liquid or the like to a light-passage.

### BACKGROUND

Fiber-optic cannulas were known. Such devices had a form of ferrule or body in which an optical fiber was held with a free end often protruding from an implant end thereof, in a manner that when the implant end of the cannula was placed into contact with a surface of the body, the optical fiber held therein protruded into the body tissue to emit light therein.

Optical fibers are delicate things, having specific characteristics. One of these is the fact that they should not be bent along less than a critical radius of curvature, lest the intensity of the light signal be dissipated externally.

Henceforth, designing a fiber-optic cannula comes with design restraints related to the nature of optical fibers.

There was a need for an improved cannula which would allow not only to implant a light signal into the body tissue, but also a liquid, electrical wires or the like.

### SUMMARY

In accordance with one aspect, there is provided a hybrid fiber-optic cannula comprising: a body having an implant end, a light passage extending through the body, and a light inlet end coinciding with the light passage, an optical fiber held in the body in coincidence with the light passage and oriented out the implant end, the light inlet end being opposite the implant end relative to the light passage, and a conduit extending through the body between a conduit outlet located at the implant end and a conduit inlet.

In accordance with another aspect, there is provided a method of making a hybrid cannula, the method comprising: providing a body having an implant end, a light passage extending through the body, and a light inlet end coinciding with the light passage, the light inlet end being opposite the implant end relative to the light passage, a cylindrical bore extending into the body from the light inlet end and leading to a stop, and a conduit extending through the body between a conduit outlet located at the implant end and conduit inlet; providing an optical fiber hub having a cylindrical shape and a central aperture receiving an optical fiber held in the central aperture; and inserting the optical fiber hub into the cylindrical bore, into abutment with the stop, with the optical fiber held in the body in coincidence with the light passage and having a tip protruding out the implant end as an extension to the light passage.

Many further features and combinations thereof concerning the present improvements will appear to those skilled in the art following a reading of the instant disclosure.

### DESCRIPTION OF THE FIGURES

In the figures,

FIG. 1 is an oblique view of an example of a hybrid cannula;

FIG. 2 is a cross-sectional view of the hybrid cannula of FIG. 1;

FIG. 3 is a cross-sectional view of an other example of a hybrid cannula;

FIG. 4 is a cross-sectional view of still another example of a hybrid cannula;

FIG. 5 is an oblique view of yet another example of a hybrid cannula; and

FIG. 6 is a cross-sectional view of the hybrid cannula of FIG. 5.

### DETAILED DESCRIPTION

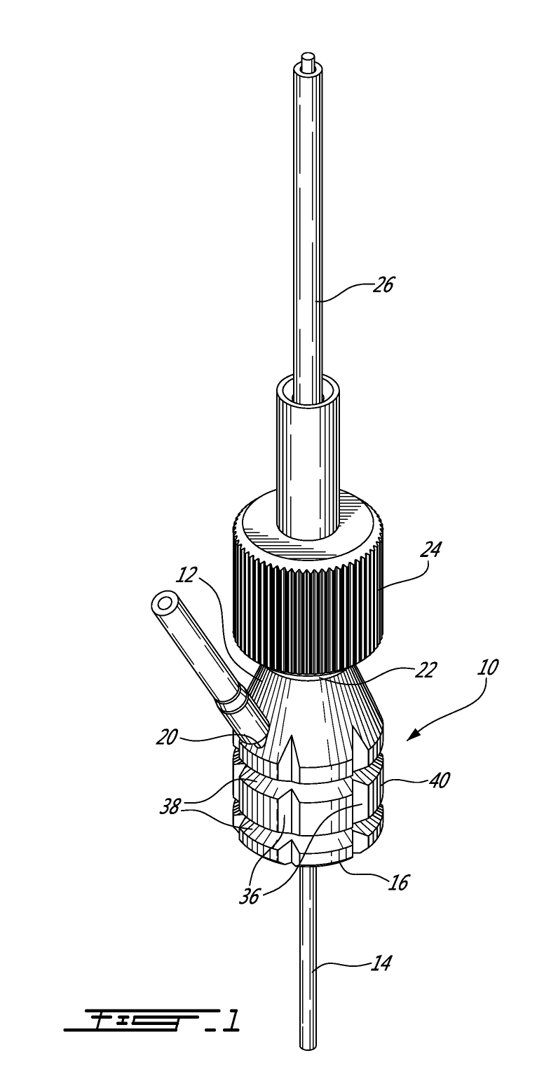

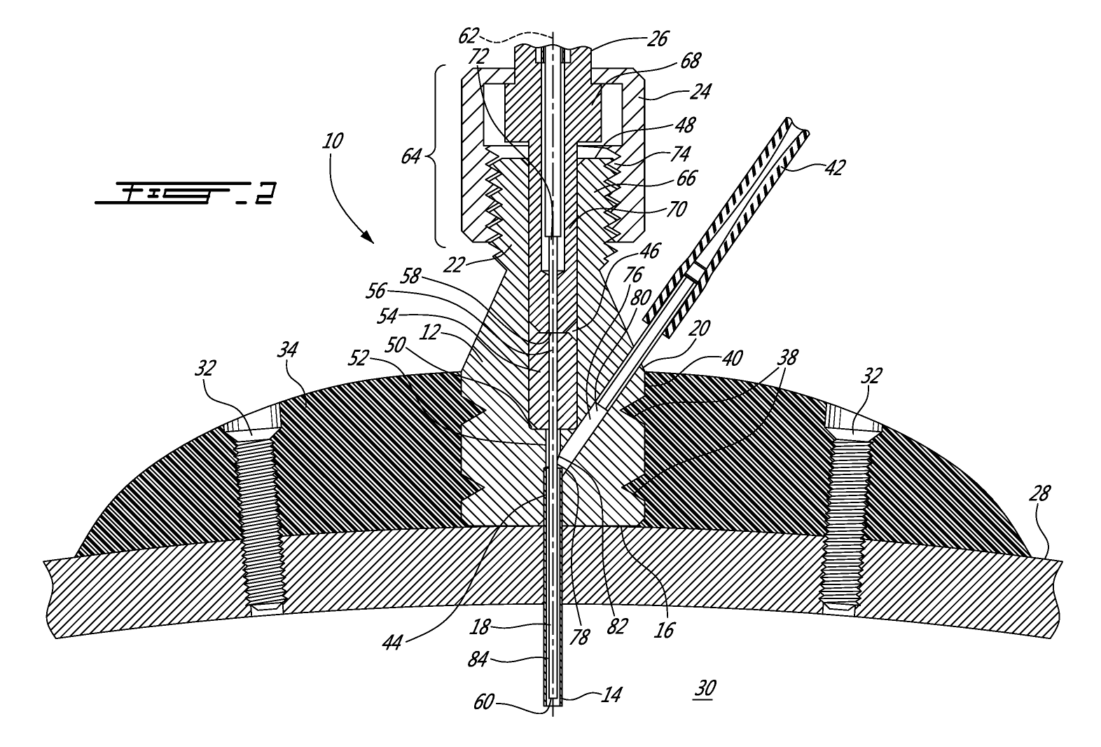

FIGS. 1 and 2 show an example of a hybrid cannula 10. The hybrid cannula 10 can be seen generally to include a body 12 having a implant tube 14 extending from an implant end 16 thereof, and an optical fiber 18 (seen in FIG. 2) extends in the implant tube 14. Opposite the implant end 16, the body 12 has conduit connector portion 20 and a fiber optic connector member 22. The fiber optic connector member 22 is shown here connected with a mating connector member 24 of a fiber optic patch cord 26.

In FIG. 2, the hybrid cannula 10 is shown in use, with the implant end 16 held against body surface 28, with the implant tube 14 and optical fiber 18 extending into the body tissue 30. In this embodiment, the body surface 28 is that of a skull and the body tissue 30 includes brain cells. To keep the hybrid cannula 10 secured to the skull, medical screws 32 are used in the skull adjacent the hybrid cannula 10, and a bonding material 34, such as an epoxy resin for instance, holds the hybrid cannula 10 in relation with the medical screws 32, thereby maintaining the hybrid cannula 10 secured to the skull. As better seen in FIG. 1, both radial grooves 36 and tangential grooves 38 are formed in the surrounding surface 40 of the hybrid cannula 10 to provide surface irregularities which allow better retention with the bonding material 34.

FIG. 2 shows additional detail about the hybrid cannula 10 and the connections with the fiber optic patch cord 26 and a conduit tube 42. More particularly, in this particular example, a bore, which can be referred to as the outlet bore 44, extends inwardly from the implant end 16 of the body 12. In this embodiment, the outlet bore 44 is sized specifically to snugly receive the implant tube 14. An other bore, which can be referred to as the light inlet bore 46, extends inwardly from a light inlet end 48 of the body 12, down to a stop 50. An aperture 52 is provided in the stop 50 by which the light inlet bore 46 communicates with the outlet bore 44. As shown, an optic fiber hub 54 having an external diameter corresponding to the diameter of the light inlet bore 46 is held therein, in abutment against the stop 50. The optic fiber hub 54 holds the optical fiber 18 securely held in an aperture 56 therein. The optical fiber 18 has an inlet tip 58 which coincides with a flat inlet end of the hub 54, and an outlet tip 60 which extends out the other side of the hub 54, through the aperture 52 and out the implant end 16 of the body 12, into the body tissue 30. For assembly, the optical fiber 18 can first be assembled to the hub 54, the hub 54 can then be positioned into the light inlet bore 46 with the optical fiber 18, into abutting contact with the stop 50, and the implant tube 14 can then be engaged with the outlet tip 60, slid along the optical fiber 18, and secured to the outlet bore 44.

A light passage 62 can thus be said to extend across the body 12, successively along the light inlet bore 46, the aperture 52, and the outlet bore 44. The light passage 62 can be straight as shown, in which case the bores 44, 46 and the aperture 52 can be machined using conventional machining tools. The optical fiber 18 held in the hub 54 coincides with the light passage 62. In this embodiment, the cannula 10 includes an optic fiber connector 64 which allows the cannula 10 to be disconnected from a light source and/or detector and reconnected at will. In this embodiment, the connector 64 includes a male threaded portion 66 on an outer surface of the body, and a female bore coinciding with the light inlet bore 46. The patch cord 26 includes a ferrule 68 with a male member 70 matching the female bore of the cannula 10 for sliding engagement therewith into abutment with the hub 54, at which point an optical fiber 72 of the patch cord 26 is positioned in optical connection with the optical fiber 18 held by the hub 54, and the optical fiber 72 of the patch cord 26 can be said to coincide with the light passage 62. A threaded female member 74 of the patch cord 26 can be threadingly engaged with the threaded male member 66 of the cannula body 12 to hold the male member 70 against the hub 54 and thereby maintain a satisfactory optical connection. In an alternate embodiment, magnets can be used instead of a threaded engagement, for instance.

A conduit 76 is also formed inside the cannula body 12. In this embodiment, the conduit 76 branches off from the light passage at a split point 78 to a side of the cannula body 12, and is in fluid communication with the outlet bore 44. More particularly, in this specific embodiment, a conduit bore 80 extends obliquely from a side of the cannula body 12 into an inner region 82 located below the hub 54. The implant tube 14 has an internal diameter which is larger than an external diameter of the optical fiber 18, thereby forming a spacing 84 which forms an extension to the conduit 76, extending between the inner region 82 of the conduit 76 below the hub 54 and into the body tissue 30 adjacent the optical fiber 18. This can allows injection of a fluid substance in the immediate periphery of the optical fiber tip 60 in the body tissue 30, for instance. The fluid substance is constrained to the conduit first by the conduit tube 42, then by the conduit bore 80, then by the internal walls of the inner region 82, and then collectively by the external surface of the optical fiber 18 and the internal wall surface of the implant tube 14.

Both the hub 54, the optical fiber 18 in the hub 54, and the implant tube 14 can be held in place with a sealing adhesive, which prevents the passage of the fluid elsewhere than along the conduit extension spacing 84. In alternate embodiments, the depth of the implant tube 14 and of the optical fiber 18 can vary independently from one another. Further, in still other embodiments, something else than a fluid can be conveyed in the conduit 76, such as an electrical wire or the like, for instance.

The embodiment described above with reference to FIGS. 1 and 2 is particularly suited for use on laboratory rats where the cannula 10 can be permanently bonded to the skull of the rat while the fiber optic patch cord 26 and conduit tube 42 can be connected thereto for experiments, or disconnected at will. Once disconnected, the laboratory rat is left with only the cannula body 12 on his skull and can thus move with a high degree of freedom compared to embodiments where the cannula would not have such ready connection means.

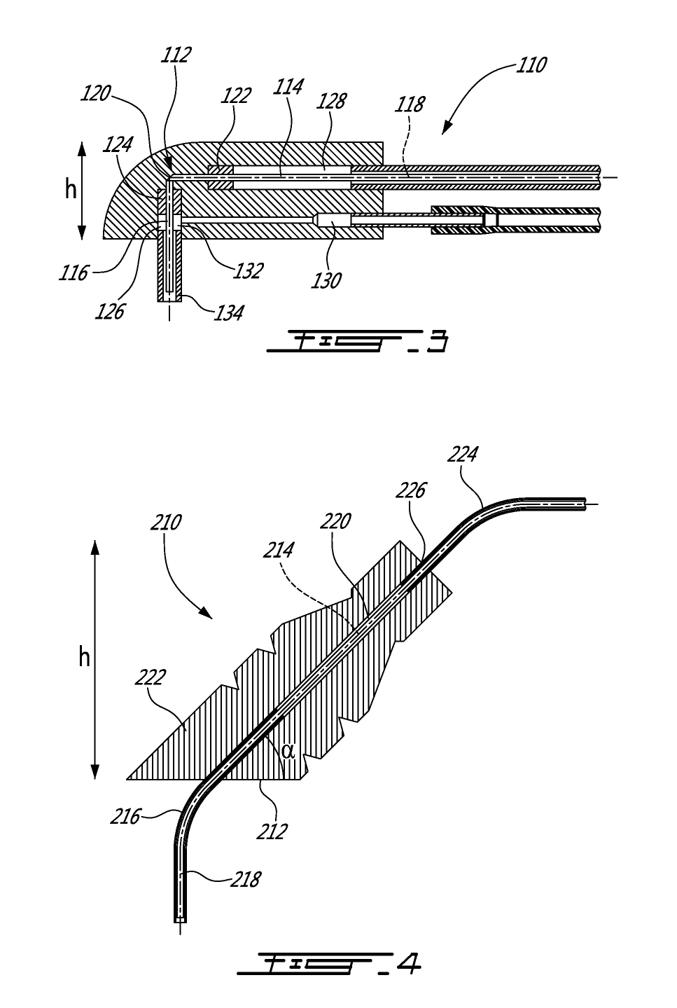

FIG. 3 shows another embodiment of a hybrid cannula 110 which is shown in the form of a more permanent attachment. This can be useful for injecting a light signal into a patient suffering from diabetes for instance, in order to assess information about the blood using fiber optics for instance, in addition to allowing injection of medicine. If a cannula 110 is destined for attachment during a long period, it can be practical to limit the distance h by which it protrudes from a body surface. To this end, because optical fibers can only be bent to a certain limit (determined by the critical radius of curvature) prior to suffering from substantial power losses, an angular reflector 112 can be used inside the body 114 of the cannula 110, between two sections 114, 116 of optical fiber which are each individually kept relatively straight, to allow a sharp change of direction in the light passage 118. The angular reflector 112 can be in the form of a separate mirror device or prism being positioned between internally facing tips of the optical fiber sections, for instance, or, as shown in the figure, can be in the form of a mirror being formed on an inclined portion 120 at the tip of the first optical fiber portion 114, to name two possibilities. In the embodiment shown, each optical fiber portion 114, 116 is shown held in a corresponding hub 122, 124 held in a straight bore 126, 128. A straight conduit bore 130 is also provided in fluid communication with the inner region 132 in fluid communication with the implant tube 134. Straight bores are easily machinable in a cannula body made of a material such as metal or plastic. Alternately, more complex shapes can be formed by moulding the cannula body. The hubs 122, 124 can be formed of materials such as metal, glass, or ceramics such as zirconia for instance, to name a few example. The implant tube 134 can be rigid, such as of a material like metal or medical plastic for instance, forming a needle, or can be slightly flexible depending of the application. The implant tube 134 is optional and can be entirely omitted in certain embodiments.

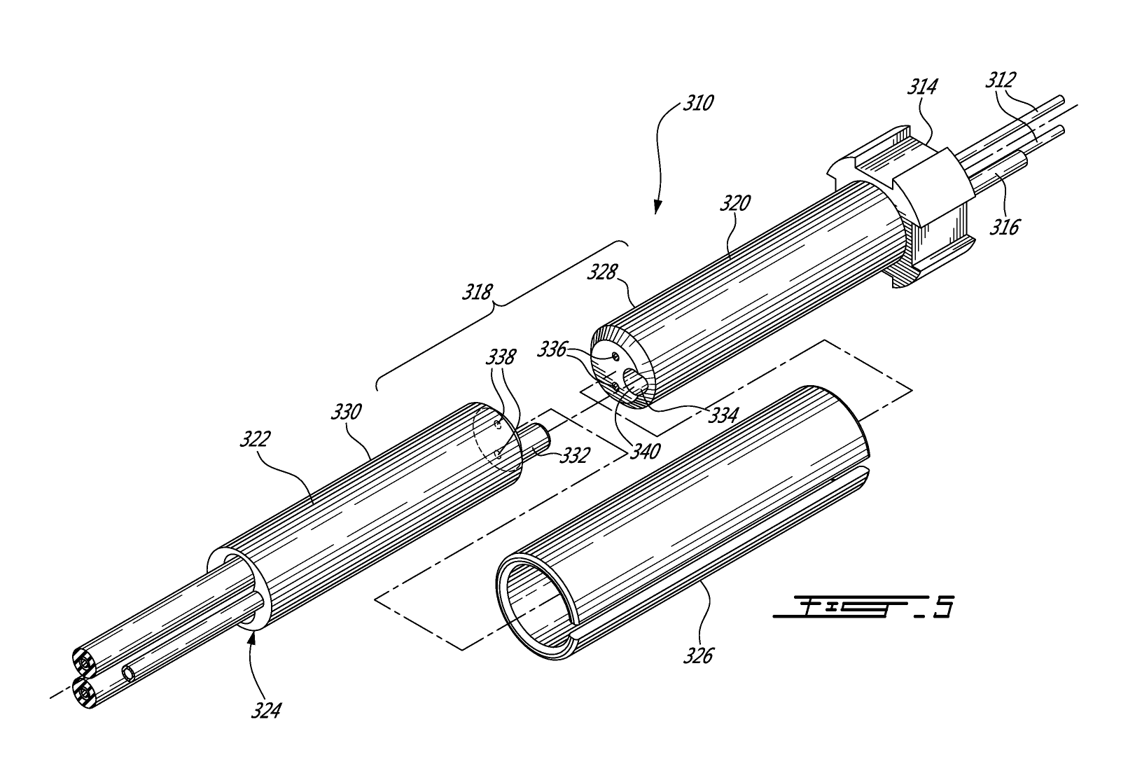

FIG. 4 shows an other embodiment of a hybrid cannula 210 in which the protruding distance h from the body surface is reduced partially by inclination of the implant end surface 212 relative to the light passage 214 by an angle a. In such an embodiment, the implant tube 216 can be curved to a certain extent, preferably within the critical radius of curvature of the optical fiber 218. The light passage 214 of the optical fiber 218 inside the body can also be curved to a certain degree if desired.

Further, FIG. 4 shows an example of a hybrid cannula 210 where the light passage 214 extends inside the conduit 220 across the cannula body 222. A tube 224 having the optical fiber 218 therein is snugly fit inside the inlet end 226 of the conduit 220. The optical fiber 218 can be left free inside the conduit 220, or can be held secured therein. The optical fiber 218 can be held in the conduit 220 by a hub having fluid apertures in addition to an optical fiber aperture, for instance (not shown).

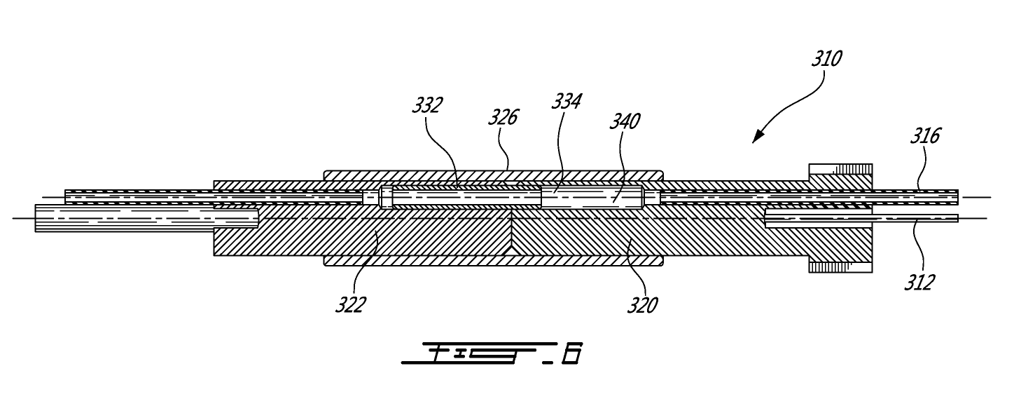

FIG. 5 shows an example of a hybrid cannula 310 having more than one optical fiber 312 protruding from the implant end 314, and having an implant tube 316 (and conduit) which is distinct from the optical fibers 312. In this particular embodiment, the hybrid cannula 310 has a sleeve connector 318. The sleeve connector 318 is formed with a ferrule 320 of the cannula 310, a ferrule 322 of the patch cord 324, and a sleeve 326 which snugly fits around male cylindrical portions 328, 330 along both ferrules 320, 322. One of the two ferrules 320, 322 has a guide pin 332 and the other one has a female bore 334 sized to receive the guide pin 332. The guide pin 332 can be off-centered in a manner to provide an angular alignment feature for the optical fiber tips 336, 338 in collaboration with the action of the sleeve 326. In this embodiment, the guide pin 332 is hollow and forms a conduit together with the female bore 334 in the other ferrule 320, as shown more precisely in the cross-sectional view of FIG. 6.

It will be understood that only some examples are provided above and illustrated in the attached figures, and that many variants are possible. For instance, in some embodiments, the optical fiber can protrude only slightly from the implant end of the cannula, be flush therewith, or even slightly recessed.

The examples described above and illustrated are intended to be exemplary only. The scope is indicated by the appended claims.

What is claimed is:

1. A hybrid fiber-optic cannula comprising: a body having an implant end, a light passage extending through the body, and a light inlet end coinciding with the light passage, an optical fiber held in the body in coincidence with the light passage and oriented out the implant end, the light inlet end being opposite the implant end relative to the light passage, and a conduit extending through the body between a conduit outlet located at the implant end and a conduit inlet.

2. The hybrid cannula of claim 1 further comprising a implant tube having an inlet end opposite an outlet end, the inlet end being held in the body in coincidence with the conduit and the outlet end protruding from the implant end as an extension to the conduit.

3. The hybrid cannula of claim 2 wherein the optical fiber protrudes out the implant end as an extension to the light passage, the implant tube has an internal diameter greater than an external diameter of the optical fiber, the optical fiber extends inside the implant tube, forming a conduit extension spacing between the external diameter of the optical fiber and the internal diameter of the implant tube.

4. The hybrid cannula of claim 2 further comprising an implant end cylindrical bore extending into the body from the implant end, the implant tube extending into the cylindrical bore to a portion of the conduit.

5. The hybrid cannula of claim 4 wherein the optical fiber extends across the portion of the conduit.

6. The hybrid cannula of claim 1 further comprising a light inlet cylindrical bore extending into the body from the light inlet end and leading to a stop.

7. The hybrid cannula of claim 6 further comprising an optical fiber hub having a cylindrical shape and a central aperture receiving the optical fiber held therein, the optical fiber hub being held in the light inlet cylindrical bore, in abutment against the stop, the optical fiber having a polished tip at one end of the optical fiber hub, opposite the implant end.

8. The hybrid cannula of claim 2 wherein the body has straight passage extending between the outlet end and a light inlet end coinciding with the light passage and comprising in succession a light inlet bore extending from the light inlet end, an intermediate section narrower than the light inlet bore, and an outlet bore extending inwardly from the outlet end, the implant tube extending into the outlet bore.

9. The hybrid cannula of claim 8 further comprising a conduit bore extending from the conduit inlet into fluid flow communication with implant tube at or near the intermediate section, the conduit bore branching away from the straight passage in an inclined manner.

10. The hybrid cannula of claim 1 wherein the body has a fiber-optic connector member at the light inlet end for connecting a corresponding fiber-optic connector member of a fiber optic patch cord for feeding light to the optical fiber.

11. The hybrid cannula of claim 10 wherein the fiber-optic connector member of the body has a male threaded portion and a female bore coinciding with the light passage.

12. The hybrid cannula of claim 10 wherein the fiber-optic connector member of the body has a male cylindrical portion defining a sleeve engagement path and a female bore defining a pin reception path.

13. The hybrid cannula of claim 12 wherein the female bore has the conduit inlet.

14. The hybrid cannula of claim 1 further comprising an angular reflector inside the body causing a change of orientation between two straight sections of the light passage inside the body.

15. The hybrid cannula of claim 1 wherein the conduit inlet does not coincide with the light passage.

16. The hybrid cannula of claim 1 wherein the conduit is sealed between the conduit inlet and the conduit outlet, for conveying a liquid therein.

17. The hybrid cannula of claim 1 wherein an outer surface of the body, adjacent the implant end, is notched to provide an improved grip to a surrounding adhesive when in use.

18. A method of making a hybrid cannula, the method comprising :
providing a body having an implant end, a light passage extending through the body, and a light inlet end coinciding with the light passage, the light inlet end being opposite the implant end relative to the light passage, a cylindrical bore extending into the body from the light inlet end and leading to a stop, and a conduit extending through the body between a conduit outlet located at the implant end and conduit inlet;
providing an optical fiber hub having a cylindrical shape and a central aperture receiving an optical fiber held in the central aperture; and
inserting the optical fiber hub into the cylindrical bore, into abutment with the stop, with the optical fiber held in the body in coincidence with the light passage and having a tip protruding out the implant end as an extension to the light passage.

19. The method of claim 18 further comprising
inserting the protruding tip of the optical fiber inside a first end of a implant tube and sliding it along a length of the implant tube; and
securing the first end of the implant tube to the implant end of the body, in fluid flow communication with the conduit.

20. The method of claim 18 wherein the securing of the first end of the implant tube is done with an adhesive, further comprising securing the optical fiber hub into abutment with the stop using an adhesive.
